(19) United States
(12) Patent Application Publication
(10) Pub. No.: US 2025/0003962 A1
(43) Pub. Date: Jan. 2, 2025

(54) DETECTION OF GLYCATED ALBUMIN IN DIABETES-AFFECTED BIOLOGICAL SAMPLES

(71) Applicant: InnSight Technology, Inc., Pittsburgh, PA (US)

(72) Inventors: Leanne Labriola, Pittsburgh, PA (US); Dipanjan Pan, State College, PA (US)

(73) Assignee: InnSight Technology, Inc., Pittsburgh, PA (US)

(21) Appl. No.: 18/741,319

(22) Filed: Jun. 12, 2024

Related U.S. Application Data
(60) Provisional application No. 63/472,579, filed on Jun. 12, 2023.

(51) Int. Cl. G01N 33/543; G01N 33/68

(52) U.S. Cl. CPC G01N 33/5438; G01N 33/6893; G01N 2333/76; G01N 2800/042

(57) ABSTRACT

Biosensors for the detection of at least one analyte, e.g., glycated albumin, and osmolarity in a biological sample are provided, The biosensors can comprise a carbon printed layer, a layer of a ligands for the one or more analytes in contact with the carbon printed layer; at least one sensor electrode; and a substrate.

### PRIORITY

This application claims the benefit of U.S. Ser. No. 63/472,579, which was filed on Jun. 12, 2023, and is incorporated by reference herein in its entirety.

### GOVERNMENT FUNDING

This invention was made with government support under grant P30DA018310 awarded by the National Institute on Drug Abuse. The government has certain rights in the invention.

### SEQUENCE LISTING

The instant application contains a Sequence Listing which has been submitted electronically in XML format and is hereby incorporated by reference in its entirety. Said XML copy, created on Aug. 15, 2024, is named 755330_IGT-001_SL.xml and is 24,933 bytes in size.

### BACKGROUND

Diabetes is a multi-organ disease caused by insufficient or absent secretion of insulin or reduced sensitivity of tissue to insulin.^{1,2} The presence of either insulin deficiency or insulin resistance in diabetes produces an abnormal rise in blood glucose levels, leading to damage to blood vessels in retina, kidneys, and nerves, therefore leading to a number of detrimental health effects such as retinopathy, nephropathy and neuropathy respectfully.³

Glycated hemoglobin (HbA1c) measurement is considered as a gold standard method for diagnosing diabetes and monitoring long-term blood sugar levels.⁵ However, HbA1C measurements can be inaccurate in patients with end-stage renal disease (ESRD), iron deficiency, or anemia, leading to lower-than-expected HbA1c levels even with increased blood glucose.⁶ Several studies suggest the challenges of HbA1c-based monitoring as their values may be adversely influenced by hemolytic or renal anemia and liver cirrhosis, and by the reduction in the life-span of erythrocytes.⁷

Alternative methods of diagnosing diabetes and monitoring blood sugar levels are needed in the art.

### BRIEF DESCRIPTION OF THE FIGURES

FIG. 1 shows an example of a biosensor chip.

FIG. 2 shows the workflow diagram that shows the correlation analysis of glycated albumin between plasma and tear film in diabetic patients.

FIG. 3 shows K_{(+162.0528)}QTALVELVK (SEQ ID NO:1), KQTALVELVK (SEQ ID NO:1), and QTALVELVK (SEQ ID NO:2) MS/MS spectra. High confidence m/z assignments of the generated fragments based on almost complete sequence coverage by the y fragment ion series, confirming their presence in both plasma and tear film.

FIG. 4A-4B shows relative quantification of glycated albumin level between plasma and tear film. The box plots (4A) compared the diabetic and non-diabetic participants in both plasma and tear, Each plot represents each data point and asterisks **depict statistical differences among groups (P≤0.01) The linear regression plot (4B) represents the strong correlation (Pearson's r=0.92019 with a P-value 0.0012) between plasma GA and tear film GA in 8 diabetic participants,

FIG. 5 shows GA level reflects the number of glycated sites. Box-and-whisker plots compared number of glycated sites in diabetes and non-diabetes for both plasma and tear. Each plot represents the mean (square), median (solid line), 25th percentile (lower limit of box), 75th percentile (upper limit of box), and outlier (upper whisker). Asterisks ** depict statistical differences among groups (P≤0.01). Filter criteria: Peptide: −10 logP>20, PTM score>20.

FIG. 6 shows correlation between other glycation sites and K525 in plasma samples. The linear regression plot represents the GA level correlation (Pearson coefficient 0.7987 with a P-value 0.00021) between K136/K137 and K525 in both diabetic and non-diabetic.

### SUMMARY

An aspect provides a biosensor for the detection of glycated albumin in a biological sample. The biosensor can comprise a carbon printed layer, a layer of a ligands that can specifically bind glycated albumin in contact with the carbon printed layer; at least one sensor electrode; and a substrate. The at least one sensor electrode can be between the carbon-printed layer and the layer of a ligand for the analyte. The ligands can be antibodies or aptamers that can specifically bind glycated albumin. Osmolarity can be detected in combination with glycated albumin. The biosensor can be configured to accept a sample size of 1-5 μL.

Another aspect provides a method for detecting glycated albumin in a sample and osmolarity of the sample. The method can comprise contacting a biosensor described herein with the sample and detecting the glycated albumin and the osmolarity with the detector. The detector can detect binding or interaction of the glycated albumin and the ligands due to a change in electrical impedance caused by binding or interaction of the glycated albumin in the sample with the ligands. The sample can be tears, tear film, aqueous layer of the tear film, aqueous humor, sweat, blood, serum, plasma, urine, saliva, or other bodily fluids. The sample size can be between about 1 μL and 5 μL.

Another aspect provides a method for detecting glycated albumin in a sample and osmolarity of the sample. The method can comprise contacting the sample with a biosensor as described herein, and inserting the biosensor into or onto a device comprising (a) a detector to measure change in electrical impedance, and (b) a screen for visualizing the change in impedance, thereby detecting one or more analytes in the sample and the osmolarity of the sample. The amount of the glycated albumin in the sample can be detected.

Yet another aspect provides a method for detecting glycated albumin in a sample and osmolarity of the sample. The method can comprise contacting the sample with a biosensor as described herein, wherein the biosensor is present in a handheld device comprising (a) a detector to measure change in electrical impedance, and (b) a screen for visualizing the change in impedance, thereby detecting one or more analytes in the sample and the osmolarity of the sample. The amount of glycated albumin in the sample can be detected.

An aspect provides a method for diagnosing diabetes. The method can comprise contacting a biosensor as described herein with a tear, tear film, aqueous layer of the tear film, or aqueous humor sample of a subject, detecting an amount glycated albumin in the sample and osmolarity of the sample, and diagnosing diabetes in the subject where the concentration of the glycated albumin in the sample is elevated as compared to a control sample or control standard and/or wherein the osmolarity of the sample is elevated as compared to a control sample or control element. An increased amount of osmolarity as compared to a control can indicate a diagnosis of diabetes. The method can further comprise treating the subject with bolus insulin, rapid-acting insulin, inhaled insulin, short-acting insulin, background insulin, intermediate-acting insulin, long-acting insulin, insulin glargine, insulin degludec, premixed insulin, biguanides, DPP-4 inhibitors, GLP-1 receptor agonists, sodium-glucose cotransporter-2 (SGLT2) inhibitors, insulin secretagogues, meglitinides, thiazolidinediones, vascular endothelial growth factor inhibitors, photocoagulation laser treatment, panretinal photocoagulation laser treatment, vitrectomy, or a combination thereof.

In any of the methods, a sample can be obtained from a subject having end-stage renal disease, liver cirrhosis, iron deficiency, or anemia. The anemia can be hemolytic anemia or renal anemia. A sample can be obtained from a subject is not required to be fasted.

Another embodiment provides a handheld device comprising a biosensor comprising a carbon printed layer, a layer of a ligands specific for glycated albumin in contact with the carbon printed layer; at least one sensor electrode; and a substrate; and a detector connected to a data acquisition system. The detector can be a multimeter. The data acquisition system can be a computer, a cell phone, or a tablet. The device can further comprise a screen that allows for visualization of an amount of the glycated albumin and an osmolarity value for the sample.

### DETAILED DESCRIPTION

Albumin is a negatively charged protein with small size (66 kDa). As the main plasma protein, albumin represents about 60% of the total proteins in the blood.⁴ Due to its relatively long half-life, serum albumin is highly sensitive to post-translational modifications (PTM), particularly glycation. In fact, serum albumin has 85 glycation sites, which include 59 lysine and 24 arginine residues.³

Human tear fluid has a complex, multilayered film structure comprising of an extensive aqueous layer sandwiched between a mucin layer and a lipid layer. The aqueous layer, primarily secreted from the lacrimal gland, contains proteins that are locally synthesized as well as those from other sources, including serum-derived proteins such as albumin, This unique tear film forms a thin, approximately 3 μm thick, and 3 μl in volume fluid layer that covers the outer mucosal surfaces of the eye, serving as the interface between the ocular surface and the environment. Glycated albumin (GA) has not been reported in terms of the correlation of its level between plasma and tear films in diabetes.

Differently from HbA1c long-term formation (about 120 days), GA is formed in a period of approximately 2 to 3 weeks. This feature enhances GA sensitivity to the rapid alterations in glucose levels, and GA should be used to screen for diabetes mellitus (DM) in diabetic patients in cases where special conditions interfere with A1c results.

Similar to other low volume biological samples, there are many challenges for testing biomarkers in tears. The current standard of care uses lateral flow assays. However, lateral flow assays are particularly affected by low volume, low concentration, and variable viscosity and this interferes with result accuracy in tear film analysis, The requirement to dilute a sample for lateral flow assays, e.g., a tear sample, can result in variability in testing that is seen in current available modalities New reliable, point-of-care methods are needed in the art for the detection of analytes in low volume and variable samples, particularly ones that can provide quantitative results.

Surprisingly, the compositions and methods described herein can accurately measure glycated albumin concentration and osmolarity in very low sample sizes, e.g., 1 to 5 μL for most adults, which is the total volume of tear film generally available in clinic for adults,

### Glycated Albumin

Methods and devices provided herein can be used for the detection of glycated albumin within a biological sample, such as tear film or saliva samples. Devices and methods as described herein can be used to detect and quantify glycated albumin, which can be elevated in amount in subjects having diabetes as compared to healthy subjects, Osmolarity of a sample can also be concurrently detected.

In one aspect, glycated albumin is present in a biological sample such as tears, tear film, aqueous layer of the tear film, aqueous humor, sweat, blood, serum, plasma, urine, saliva, or other bodily fluids.

A ligand is a substance that forms a complex with an analyte such as glycated albumin through, for example, ionic bonds, hydrogen bonds, Van der Waals forces, covalent bonds, non-covalent bonds, electrostatic interactions, rr-effects, or hydrophobic effects.

A ligand can be immobilized to the sensor (e.g., to the carbon layer and/or the sensor or working electrode) using any suitable technology, e.g., covalent coupling or capture coupling. Immobilization of one or more binding ligands onto a biosensor chip is performed so that the ligand will not be washed away by rinsing procedures, and so that its binding to analytes in a test sample is unimpeded by the biosensor chip surface. Several different types of surface chemistry strategies have been implemented for covalent attachment of ligands to biosensor chips. Surface preparation of a biosensor chip so that it contains the correct functional groups for binding one or more ligands can be an integral part of the biosensor chip manufacturing process.

One or more ligands can be attached to a biosensor chip surface by physical adsorption (i.e., without the use of chemical linkers) or by chemical binding (i.e., with the use of chemical linkers). Chemical binding can generate stronger attachment of specific binding substances on a biosensor chip surface and provide defined orientation and conformation of the surface-bound molecules.

Several examples of chemical binding include, for example, amine activation, aldehyde activation, and nickel activation. These surfaces can be used to attach several different types of chemical linkers to a biosensor chip surface. While an amine surface can be used to attach several types of linker molecules, an aldehyde surface can be used to bind proteins directly, without an additional linker. A nickel surface can be used to bind molecules that have an incorporated histidine (“his”) tag.

The interaction of ligands with their analytes (e.g., glycated albumin) can be characterized in terms of a binding affinity. For example, a ligand can bind an analyte with a K_{d} equal to or less than about 10⁻⁷ M, such as but not limited to, 0.1-9.9×10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein. A ligand can bind an analyte with an off rate (K_{off}) of less than or equal to 0.1-9.9×10⁻³ sec⁻¹, 10⁻⁴ sec⁻¹, 10⁻⁵ sec⁻¹, 10⁻⁶ sec⁻¹, 10⁻⁷ sec⁻¹. A ligand can bind an analyte with an on rate (Kₒₙ) greater than or equal to 0.1-9.9×10³M⁻¹ sec⁻¹, 10⁻⁴ M⁻¹ sec⁻¹, 10⁵ M⁻¹ sec⁻¹, 10⁶ M⁻¹ sec⁻¹, 10⁷ M⁻¹ sec⁻¹, 10⁸ M⁻¹ sec⁻¹.

A ligand for glycated albumin can be, for example, an antibody (or specific binding portion thereof) that specifically binds glycated albumin, an antibody fraction, a nucleic acid, a protein, a peptide, a peptidomimetics, an ion, a small molecule, an enzyme, or an aptamer.

An antibody or specific binding fragment thereof can be independently selected from fragments such as scFv, Fab, Fab′, Fv, F(ab′)₂, a minibody, a diabody, a triabody, a tetrabody, a tandem di-scFv, a tandem tri-scFv, an immunoglobulin single variable domain (ISV), such as, a V_{HH} (including humanized V_{HH}), a camelized V_{H}, a single domain antibody, a domain antibody, or a dAb). Antibodies can be a polyclonal antibodies, monoclonal antibodies, or specific binding fragments derived from monoclonal or polyclonal antibodies. Antibody specific binding fragments are one or more portions of an intact antibody that comprises the antigen binding site or variable region of an intact antibody, where the portion is free of the constant heavy chain domains of the Fc region of the antibody. An antibody described herein can be any class of antibody that includes, for example, IgG, IgM, IgA, IgD and IgE.

The term “specifically binds.” “binds specifically to,” “specific for” or the like, means that the antibody, a specific binding fragment thereof, an aptamer, or other suitable ligand as described below forms a complex with an antigen or polypeptide that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind include, for example, equilibrium dialysis, surface plasmon resonance, a KinExA assay, and the like.

Glycated albumin can also be detected using aptamers that specifically bind glycated albumin. Aptamers are three-dimensional oligonucleotides (RNA or single-stranded DNA structures) that can bind with high selectivity and specificity to analytes due to their spatial conformation, In some aspects, aptamers can provide for advantages over the use of antibodies, including greater thermal stability which extends their shelf-life and aptamer conformation-recovery properties that make aptamer-based sensors reusable.

Antibodies that specifically bind glycated albumin include, for example ATCC HB 9596, Abnova's monoclonal antibody (M02), clone 1A11, Ethos Biosciences monoclonal antibody A717. Apatmers that can specifically bind glycated albumin include, for example, GRA33 (see Fayazi et al., Sci Rep 10, 10716 (2020),

GABAS-01
(SEQ ID NO: 3)
ATGCGGATCCCGCGCGCAGTGCAGGGAGCC
GCTCCACGTACGTTGCGCGAAGCTTGCGC

(see Kim et al. Biosensors (Basel). 2020 Oct. 14; 10(10):141), and 5′Amino C6/TGCGGTTGTAGTACTCGTGGCCG(SEQ ID NO:4)/Thiol C6 SS 3′ (Biosearch Technologies, Petaluma, CA). Other aptamers that specifically bind glycated albumin include for example:

(SEQ ID NO: 5)
TCTATCCCCCCAGCCTTCCCACTCCAACCCTGC
(SEQ ID NO: 6)
CGGGCCGCTGCATATAACTGAATTGAATAAGCTGGTAT
(SEQ ID NO: 7)
TGGTACATCGACCATCACCGCACCTCACATATTCCGAATTACTCC
CGACGTA
(SEQ ID NO: 8)
TACATTGCTCCTGCGGAAAAATTGTCAAACCATCTACTGCGAAGC
GTGTTTT
(SEQ ID NO: 9)
TAGGAGTAGGGGGTCGTAGACGGTTGGGGCGGAACGGGCGTGGGG
CATG
(SEQ ID NO: 10)
TGGTACATCGACCATCACCGCACCTCACATATTCCGAATTACTCC
CGACGTAT
(SEQ ID NO: 11)
TCGATGGTGGGCAGCCCCAGCACATTCCGTATGTTAACCCCTGCG
TTGCCATT
(SEQ ID NO: 12)
GGTGCGGTTCGTGCGGTTGTAGTACTCGTGGCCGATAGAGGTAGT
TTCG
(SEQ ID NO: 13)
TCATACTGGGTCATGTACTTAGCTGGTCGCAGCGGGGACTGAGTT
AGTGTT
(SEQ ID NO: 14)
TCCCACGCCCGCCCGTCGTTCACCCCTCCCCGCTACCTCCCTATC
CAACTGCG
(SEQ ID NO: 15)
TCCCCCCATCACACCCAAGCCGCAGCCACCGACATAGCAAGCATT
GTCTTTCC
(SEQ ID NO: 16)
TCGGGGGGGCGTTGATTTTGTTGAAGGGAGGTATAGTGTCTGTCG
GTCTGAT
(SEQ ID NO: 17)
TCCTGCCGAACTCCAAGATCTCCGCTCCGCTCACGCTGTGTATCC
ATGGGG
(SEQ ID NO: 18)
TAGTTCTAGGCCGCCCTCGTGATAACCCCCCTCCATCTTCCCTAC
GATGTACT
(SEQ ID NO: 19)
TGGGTCATCGTCGTCTTAGGCGCGTGAAAGGGGTAGGATGGCGGG
TAGGATG
(SEQ ID NO: 20)
TGCAAGGTGGGCATTGGCATTGCGTAGCTAGGGGGTGAAGGCGTG
TGGTTTT
(SEQ ID NO: 21)
TCAGGCAAACACAATATACGCAATATCACGGTGGAATTTCAAGGC
CTTTCATCAATTGAATAAGCTGGTAT
(SEQ ID NO: 22)
TCAAAAGCGCGCTAAGCCTAGTTCGACAACTTCACCAACGACCCA
CTATTCGT
(SEQ ID NO: 23)
TCCCTAACCCGCTCTAACCAACCGCGCTCAGTCCGACATCCGTAA
ACGGGC
(SEQ ID NO: 24)
TCCAACCCAGACCAACATTCCTCGCCTCCGCTATCTGCACCGCCA
CACATAAC

See US Pat. Publ. 20160237436. Other ligands like 3-acrylamido phenylboronic acid (3-APBA) can also be used.

In one aspect a ligand for glycated albumin is stable at room temperature (about 20 to 22° C.), field temperatures (about 0 to 49° C.), refrigeration temperature (about 1 to 5° C.), or freezing temperature (about 0 to −70° C.) for 1 day, 2 days, 3 days, 4 days, 5 days, 1 week, 2 weeks, 3 weeks, 4 weeks, 2 months, 3 months, 6 months, 1 year, 2 years or longer.

### Osmolarity

Existing methods of detecting osmolarity of a sample provide a quick result, but are limited because of high error rates with the lower sample of volume from tear film collection. In a research setting, the most commonly applied laboratory technique for measurement of tear osmolarity is through observation of the change in the freezing point of tear samples, The technique, however, includes the need for specialist expertise, constant maintenance of equipment, a significant amount of time, a large laboratory setup, and there may be errors due to evaporation of the test sample or control standards. The instant compositions and methods can accurately detect osmolarity of small sample volumes (e.g., tear film) using resistance or impedance readings at a location on a biosensor chip that can be present in a point of care device as described herein,

### Biosensor Chips

A biosensor chip or biosensor can comprise an electrode printed on a carbon layer. Carbon atoms can possess various physical structures with distinct physical attributes. A carbon layer can be made up of carbon spheres and ellipsoids, carbon nanotubes (CNTs), nanohorns (CNHs), graphene, fullerene, carbon dots (CDs), carbon nanodiamonds (CNDs), graphene quantum dots (GQD), single-walled carbon nanotubes (SWNTs), multiwalled carbon nanotubes (MWCNTs), or cup-stacked carbon nanotubes (CSCNTs), graphite, and carbon nanofibers (CNFs), carbon sponges felt. A detection ligand layer (e.g., an enzyme, protein, aptamer, or antibody layer) can be present on the electrode. A barrier layer can cover the detection layer. An electrode is a composition that, when connected to an electronic device, is able to sense a current or charge and convert it to a signal. Alternatively, an electrode can be a composition that can apply a potential to and/or pass electrons to or from connected devices.

Electrodes include, but are not limited to, certain metals and their oxides, including gold; copper; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). In one embodiment, the electrode can be an interdigitated electrode, micro-interdigitated gold electrode (MICE), or a digitated electrode (e.g., a digitated gold electrode).

In one aspect, gold is deposited on a biosensor electrode at a thickness of about 50, 100, 200, 300, 400, 500, or more nm.

The electrode can be a planar electrode. The electrode can be deposited on a biosensor by a variety of methods including, but not limited to, screen-printing or evaporation. An electrode may be open or covered by a cover to form a defined volume cell.

A biosensor electrode can detect a change in resistance or impedance caused by the interaction of a ligand and an analyte (e.g., glycated albumin) such as an antibody or aptamer specific for glycated albumin. Reactance is the resistance (R) offered to AC current by inductors and capacitors, Impedance (Z) is the sum of the resistance (R) and reactance, The change in resistance or impedance can indicate an amount of the analyte present in the sample. A biosensor electrode can also detect a change in resistance or impedance caused by the osmolarity of the sample. The change in resistance or impedance can indicate an amount of osmolarity of a sample.

In an aspect, a working or detector electrode, a reference electrode, and a counter electrode can be present on a chip, A reference electrode can allow for the measurement of the potential of the working/detector electrode without passing current through it while a counter electrode can allow for passing current, A biosensor chip can be configured to fit in a device that can be used to collect a sample and analyze the sample, A biosensor chip can have a height of about 0.3, 0.4, 0.5, 0.6, 0.635, 0.7, 0.8, 1.0 mm or more. A biosensor chip can have a length of about 20, 25, 30, 35, 40, 45, 50 mm or more. A biosensor chip can have width of about 5, 6, 7, 8, 9, 10, 11, 12, 13, 14, 15 mm or more. A detector device can comprise a slot to insert a consumable (i.e., one or more biosensor chips or cartridges).

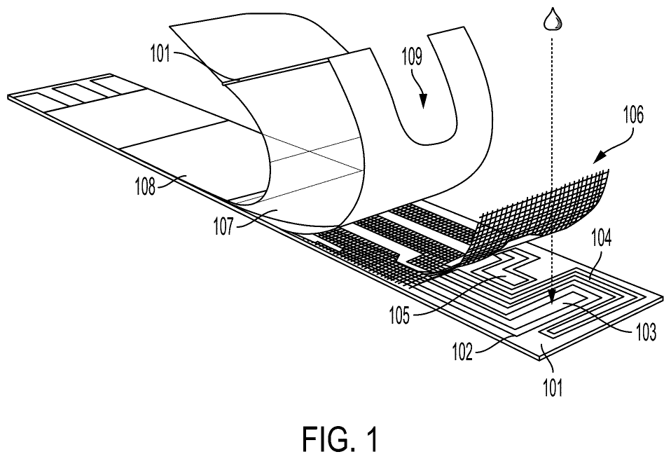

In an aspect a chip can comprise a base layer such as polyester, plastic, or polymer base. See FIG. 1. A base layer can comprise one or more of an acrylamide, cellulose, nitrocellulose, glass, indium tin oxide, silicon wafer, mica, polystyrene, or polyvinylidene fluoride (PVDF) filter, filter paper (e.g., Whatman), glass fiber filters (GF), fiberglass, polyethylimine coated GFs, porous mylar or other transparent porous films, cellulose nitrate (CN) membrane, mixed cellulose ester membrane, cellulose acetate membrane, polyethersulfone (PES) membrane, PTFE membrane, ultrafiltration membranes of poly(vinyl chloride) (PVC), carboxylated poly(vinyl chloride) (CPVC), polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamine acids. The base layer can be formed into pre-perforated strips, individual strips, individual sheets, or any other suitable shape. A carbon layer 102, e.g., a carbon printed layer can be present on the base layer 101. A working or sensor electrode 103 can be present on the carbon layer and can comprise one or more detection ligands (e.g., antibodies, enzymes, proteins, or aptamers). The one or more ligands can be layered and/immobilized on the working or sensor electrode on one or more portions of the electrode or over the entire electrode. A layer of ligand is not necessarily contiguous over the working or sensor electrode depending on the methodology used to apply the ligands to the electrode. That is, the ligands can be spaced from each other on the electrode and still be considered a layer of ligands. A reference electrode 104 can be present on the biosensor chip and can be, for example, silver and/or silver chloride. In an aspect two more resistance or impedance values can be present on the same circuit so that two of more properties of the test fluid can be tested. A fill trigger electrode 105 can also be present on the carbon layer and can be silver and/or silver chloride or other suitable material. The carbon layer can provide one or more conductive tracks or microchannels (e.g., 1, 2, 3, 4, 5 or more) for the working, reference, and trigger electrodes. A sample can be delivered to the conductive tracks or microchannels which lead to the electrodes. More than one working or sensor electrodes (e.g., 1, 2, 3, 4, 5, or more) can be present to detect and quantify more than one analyte at a time. A biosensor can comprise a detection area for the at least one analyte and a detection area for osmolarity. A detection area for the at least one analyte and the detection area for osmolarity can be located on the same sensor electrode. A detection area for the at least one analyte and the detection area for osmolarity can be located on different sensor electrodes.

A mesh layer 106 can be present on the electrodes to support transport of a biological sample to the sample measurement chamber at the working or sensor electrode. An insulation print layer 107 can hold the mesh layer in place, insulate the circuitry, and define the sample chamber. A tape layer 108 on top of the insulation print layer can provide protection from environmental and physical effects. This tape layer can be semi-opaque or clear to allow for the visual confirmation of the sample fill. An air bleed thread 110 can be present in the tape layer to allow the sample chamber to fill.

Another aspect provides a cartridge including one or more biosensor chips. A cartridge can comprise a base including an air bladder and a puncturing barb for an optional calibrant pouch (which can include any calibrants or reagents). A cartridge label can be present. One or more biosensor chips (e.g., 1, 2, 3, 4, 5, or more) can be present on the cartridge base and are covered by a tape gasket. A cartridge cover can be present on the tape gasket. A cartridge cover can comprise one or more microfluidic channels (e.g., 1, 2, 3, 4, 5 or more), a sample entry well, and a sample entry well gasket. The cartridge can comprise a sample fill mark, a tab for snap closure, and a sample well.

One or more cartridges or biosensor chips can be disposable and can fit within a sensing device or detector.

### Detector

A detector device can be classified as an electrochemical sensor, e.g., amperometric, potentiometric, impedimetric, photoelectrochemical, and electrogenerated chemiluminescence sensor. Amperometric sensors have a voltage placed between reference and working electrodes to cause electrochemical oxidation or reduction. The is current is measured as a quantitative indicator of the analyte's concentration, In potentiometric sensors when specific sensor-analyte interactions occur, a local Nernstian equilibrium is formed at the sensor interface which can be used to calculate the analyte's concentration. Impedimetric sensors or conductometric sensors measure changes in the surface impedance to detect and quantify analyte-specific recognition events on the electrode.

In an aspect, impedance based biosensor detection comprises application of a small amplitude AC voltage to the sensor electrode and measurement of the in/out-of-phase current response as a function of frequency. Impedance biosensors can be fabricated by immobilizing a ligand onto a conductive and biocompatible electrode and then detecting the change in the interfacial impedance upon analyte binding.

One or more biosensor chips or cartridges can be used with a detector device, A detector device can be handheld (i.e., less than 12 inches in length and 2, 1, 0.75, 0.5 pounds or less in weight). A detector device can be connected to a data acquisition system. A detector device can include an integrated sample collection element A detector can comprise a digital or analog multimeter that can measure voltage, current, impedance and/or resistance. A detector can also be a spectrophotometer, fluorometer, or a spectrometer like a Raman spectrometer or a Fourier transform infrared spectrometer.

A data acquisition system can be, e.g., a computer, a hand-held device, a cell phone, and/or a tablet. The detector provides information (e.g., a sample identifier, a subject identifier, a quantity detected of one or more analytes (e.g., glycated albumin), an osmolarity reading, a positive or negative reading regarding the presence or absence of an analyte, or a combination thereof) to the data acquisition system, which can then analyze the information and provide an easy to read and interpret result.

A device can further comprise a screen that allows for visualization of an amount of an analyte, such as glycated albumin, present in a sample and/or the osmolarity of a sample. A device can be battery operated, Results can be time stamped to avoid any issues with diurnal curve, The devices can be Bluetooth capable to transfer data directly into an electronic medical record.

An example of a work-flow using a device can comprise, for example, first turning a device on. A menu can be displayed including selections such as sample collection, history, and settings. Where sample collection is selected the device display instructs a user to insert a new cartridge. The device can then go through a stabilization mode in preparation for sample collection. In an aspect the stabilization is where the impedance reaches a stable state. The device display can then instruct the user to collect a sample, e.g., a tear film. Once the device receives enough sample, it can instruct the user that the sample has been collected. The device can ask the user to return the device to a stand to start analyzing the sample. The device can then analyze the sample. The device display can then show the result and show where in a range the sample falls (e.g., normal or elevated or depressed). The device can display a result in, e.g., pg or ng/mL and show how the result falls into a range. If the analysis is unsuccessful an error result can be displayed that instructs the user to retest. The device display can then indicate to the user that the cartridge can be removed. The device can then be turned off. In an aspect, a low battery screen can be displayed when the battery power is low.

Also provided are kits comprising one or more biosensors or cartridges, one or more disposable biosensors or cartridges, a detector, a data acquisition system, or combinations thereof.

### Methods of Detection of Analytes and Diagnosis

In an aspect the devices and methods provide 60% less processing time and greater sensitivity (70% compared to 30%) than other tests, In an aspect, results can be delivered in about 10, 7, 5, 4, 3, 2, or less minutes, The instant methods and devices allow for direct tear collection (without dilution) and microfluidic control. Microfluidic channels can be reduced in size to accept and process as little as 1 μL of fluid (e.g., about 50, 40, 30, 20, 10, 5, 4, 3, 2, 1, 0.5, 0.1, 0.05 μL or less). Microfluidic channels can be reduced in size to accept and process about 1 μL to about 5 μL of fluid (e.g., about 50, 40, 30, 20, 10, 5, 4, 3, 2, 1, 0.5, 0.1, 0.05 μL or less).

Methods are provided for detecting diabetes or diabetic retinopathy in a subject comprising detecting the level or amount of an analyte (e.g., glycated albumin) and/or an osmolarity level in a biological sample (e.g., tear film) from a subject. An elevated level of glycated albumin and/or an elevated osmolarity can indicate diabetes or diabetic retinopathy, is present in the subject. The level or amount of glycated albumin and/or osmolarity in a tear film can be compared to a control sample or control standard. If an elevated level of glycated albumin and/or an elevated osmolarity is present in the tear film, then a medical practitioner can administer one or more treatments (e.g., administration of medication, etc.) to the subject.

In an aspect, one portion of a biosensor chip can comprise ligands that bind glycated albumin and another portion of the biosensor chip can comprise no ligands. This portion of the biosensor chip can be used to detect osmolarity of the sample. In other aspects, two or more biosensor chips can be used, where one has ligands that bind glycated albumin and another biosensor chip does not have the ligands. This biosensor chip can be used to detect osmolarity.

In an aspect, the detection of osmolarity and/or one or more analytes (e.g., glycated albumin) as described herein can be used to detect and/or diagnose diabetes or diabetic retinopathy.

Also provided are methods of detecting an analyte such as glycated albumin and/or osmolarity in a sample comprising contacting a biosensor chip or cartridge with the sample and detecting the analyte with a detector.

The detector can detect binding of the analyte and the analyte ligand due to a change in electrical resistance or impedance caused by interaction of the analyte in the sample with the analyte ligand; due to a change in mass on the biosensor; due to a colorimetric change; due to a fluorescent reaction; due to a change in a Raman spectroscopy reading, or due to a change in a Fourier transform infrared spectroscopy reading. Osmolarity can also be detected in this manner, In one embodiment, after contacting a biosensor chip or cartridge with a sample, the biosensor is inserted into or onto a device comprising a detector to measure a change caused by interaction of an analyte in the sample and a ligand for the analyte or osmolarity such as a change in electrical resistance or impedance. A screen for visualizing the change in resistance or impedance is provided such that an analyte in the sample is detected.

In an aspect, a subject or patient is a mammal, such as a human, primate, canine, feline, equine, or bovine. In an aspect, a subject has end-stage renal disease, iron deficiency, anemia, hemolytic anemia, renal anemia, or liver cirrhosis. In an aspect, a subject is not required to fasted before testing. Fasting means no food or liquids other than water for 2, 4, 6, 8, 10 or more hours prior to testing.

In an aspect a sample is collected and tested directly without any additional preparation or dilution. In an aspect, a sample can be filtered to remove smaller proteins using, for example, a 10, 20, or 30 kDa Molecular Weight Cut Off (MWCO) filter.

The presence, absence, or an amount of an analyte (e.g., glycated albumin) in the sample optionally along with osmolarity can be detected. The amount of an analyte, such as glycated albumin, can indicate the severity of the diabetes or diabetic retinopathy. That is, the higher the amount of analyte, e.g., glycated albumin, or osmolarity the greater the severity of the disease or condition (e.g., diabetes or diabetic retinopathy), In an aspect, a device can detect about 100, 90, 80, 70, 60, 50, 40, 30, 20, 10, 5, 1, 0.1, 0.001 g/L or less of glycated albumin in a sample. In an aspect abnormal tear osmolarity can be about 308, 310, 312, 314, 316, 318, 320 or more mOsm/L in one eye, or a difference of greater than about 7, 8, 9 10 or more mOsm/L between the eyes.

Methods of diagnosing diabetes or diabetic retinopathy are provided, The methods comprise contacting a biosensor chip as described herein with a biological sample, e.g., a tear; tear film; aqueous layer of the tear film, or aqueous humor sample of a subject and detecting an amount of an analyte (e.g., glycated albumin) and/or osmolarity in the sample. Diabetes or diabetic retinopathy is diagnosed in the subject where the concentration of the analyte (e.g., glycated albumin) in the sample is elevated as compared to a control sample or control standard and/or the osmolarity is elevated as compared to a control sample or control standard.

Methods and compositions as described herein can provide specificity i.e., the ability of an assay to measure a specific analyte, e.g., glycated albumin, rather than other analytes in a sample. The specificity can be, for example, 70, 75, 80, 90, 95, 96, 97, 98; 99% or more. Methods and compositions of the invention can provide 10, 9, 8; 7, 6, 5, 4, 3, 2, 1% or less false positive results.

In one aspect, a biosensor chip can be used to detect analytes such as glycated albumin and/or osmolarity in other types of samples such as sweat, blood, serum, plasma, urine, saliva, or other bodily fluids to diagnose or detect other conditions.

Analytes such as glycated albumin can be found in higher amounts or levels in injured or diseased samples (e.g., a sample of a subject with diabetes or diabetic retinopathy) as compared to control subject samples from non-injured or non-diseased subjects. The relative levels of analytes, such as glycated albumin, in subject samples can indicate progression of disease and disease severity. That is, in some instances, a greater amount or level of analyte in a test sample means a more severe disease state or condition.

In some embodiments, the level of analytes such as glycated albumin or osmolarity in a test sample is compared the level of the analyte or osmolarity in a control sample from one or more normal control subjects. Typically, the measured control level in the control sample is then compared with the analyte level or osmolarity measured in the test sample. Alternatively, the level of an analyte such as glycated albumin or osmolarity level in the test sample is compared to a previously determined or predefined control level (a “control standard”). For example, the control standard for an analyte such as glycated albumin can be calculated from data, such as data including the levels of the analyte in control samples from a plurality of normal control subjects. The normal control subjects and the test subject under assessment can be of the same species.

### Methods of Treatment

Diabetes types 1 and 2 can be treated with mealtime (i.e., bolus) insulin such as rapid-acting insulin (e.g., glulisine (Apidra), insulin lispro (Humalog), insulin aspart (NovoLog), and inhaled insulin (Afrezza), short-acting insulin (e.g., regular (Humulin R, Novolin R); background (i.e., basal) insulin; intermediate-acting insulin (e.g. NPH (Humulin N, Novolin N), long-acting insulin (e.g., insulin detemir (Levemir), insulin glargine (Lantus, Basaglar, Toujeo), and insulin degludec (Tresiba); premixed insulin (a combination of bolus and basal insulin), and combinations thereof. Additional Type 2 diabetes medications include, e.g., biguanides (e.g., Metfomin), DPP-4 inhibitors (e.g., sitagliptin saxagliptin, and linagliptin), GLP-1 receptor agonists (e.g., exenatide, dulaglutide, lixisenatide, liraglutide and semaglutide), sodium-glucose cotransporter-2 (SGLT2) inhibitors (e.g., canagliflozin, dapagliflozin, and empagliflozin), insulin secretagogues (e.g., sulfonylureas (e.g., glimepiride, glipizide, glyburide), meglitinides (e.g., repaglinide, nateglinide), and thiazolidinediones (e.g., pioglitazone, rosiglitazone) and combinations thereof.

Diabetic retinopathy can be treated with vascular endothelial growth factor inhibitors, which are injected into the vitreous of the eye. These include, for example, faricimab-svoa (Vabysmo), ranibizumab (Lucentis), aflibercept (Eylea), and bevacizumab (Avastin), photocoagulation laser treatment (focal laser treatment), panretinal photocoagulation laser treatment, (scatter laser treatment), and vitrectomy.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used, Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure, It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above,

### Examples

### Example 1: Methods and Participants

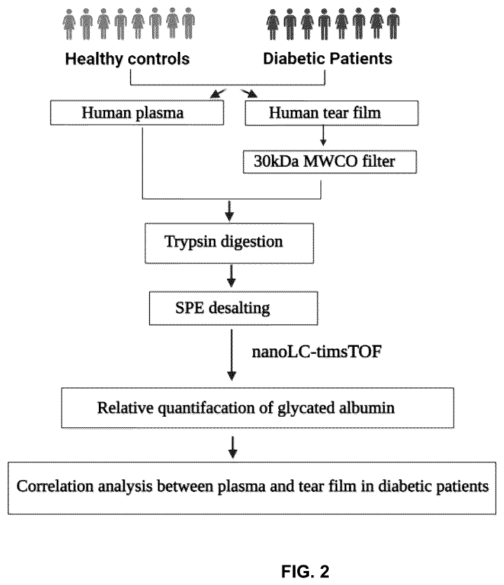

A workflow chart illustrating the study design is shown in FIG. 2. Participants>18 years of age with a diagnosis of diabetes (either Type 1 or Type 2) were compared to healthy controls. History of “pre-diabetes” or impaired glucose tolerance without a diagnosis of diabetes, or a diagnosis of severe dry eyes were excluded for the experiment. This research was conducted in compliance with an accredited Institutional Review Board (IRB). Eight healthy participants and eight participants with diabetes were admitted to Carle Hospital, Champaign, IL, and blood and tear film samples were collected.

### Example 2: Tear Film and Plasma Collection

Unstimulated tear samples were collected by an ophthalmologist with a micropipette glass capillary tube, which were placed in the tear lake that forms on the lower eyelid, and the fluid was passively obtained by capillary action without disturbing the cornea. An average of 2 μL was collected for each sample. All the tear samples were frozen after collection at −80° C. On the same day the tear film was collected, blood samples were collected. For this step, 2-3 ml of blood was collected into a heparin tube, then was centrifuged at 2500×g for 15 minutes at 4° C. to isolate the plasma from the remaining cellular debris. The plasma (supernatant) was transferred to a different tube and heat-treated at 56° C. in a water bath for 30 minutes. After the heat treatment, all the plasma samples were stored at −80° C. until laboratory measurement. All samples were transported from the ophthalmic clinic to the laboratory for testing.

### Example 3: Trypsin Digestion

An average 2 μL tear film sample was reconstituted in 500 μL LC-MS grade water, followed by loading into 30 kDa Molecular Weight Cut Off (MWCO) ultra-0.5 centrifugal filter (Amicon, Millipore Sigma). The samples were centrifuged at 14000×g for 15 min to remove the proteins and peptides below 30 kDa. The tubes were reserved then centrifuged for 1000×g for two min to collect the proteins above 30 kDa. The samples were then diluted in 100 μL Rapigest. 1 μL plasma sample was diluted in 100 μL without the MWCO filter process. Dithiothreitol (DTT) was added to the sample to a final concentration of 10 mM. The samples were then incubated at 60° C. for 30 minutes, and iodoacetamide was added to the sample to a final concentration of 15 mM. Next, samples were incubated again for 30 min at room temperature in the dark, and finally DTT was added again to a final concentration of 20 mM. The samples were incubated with the DTT-containing buffer to quench alkylation reaction for 30 min at room temperature in the dark. Sequencing Grade Modified Trypsin (Promega, Madison, WI) was added to each sample at a trypsin/protein ratio (w/w) of 1:20. The samples were digested for 16 hours in a 37° C. water bath. After the incubation, the enzyme reaction was quenched by adding 1 μL of formic acid to reduce the pH to 3-4, and then the samples were dried down completely in the Speedvac, and stored at −80° C. until further use. The dried protein digested were reconstituted in 200 μL of 0.1% formic acid in water, and they were desalted using Pierce™ Peptide Desalting Spin Columns (ThermoFisher Scientific) as per the manufacturer's instructions. The tryptic peptides were eluted from the spin columns with 600 μL of 50/50/0.1 acetonitrile/water/FA, and then the eluate was dried down completely in the speed vac and stored at −80° C.

### Example 4: Nano Liquid Chromatography-Trapped Ion Mobility Spectrometry-Time-of-Flight Mass Spectrometry Analysis

Liquid chromatography (LC) separation of the tryptic peptides was performed on a nanoElute LC system (Bruker Daltonics). Mobile phases consisted of LC-MS grade water containing 0.1% formic acid (solvent A) and LC-MS grade acetonitrile containing 0.1% formic acid (solvent B). For each sample analysis, 100 ng of the peptide digest were loaded on a nanoElute TRAP pre-column (Bruker Daltonics, 25 μm×6.0 mm, C18, 1.9 μm particles, 120 Å pore size) using solvent A for 5 min. The peptides were then separated on a nanoElute FIFTEEN column (Bruker Daltonics, 75 μm×150 mm, C18, 1.9 μm particles, 120 Å pore size). The separation was carried out at 40C with a flow rate of 400 nL/min using the following gradient: 2-10% B from 0-2 min, 10-45% B from 2-102 min, 45-60% B from 104 to 119 min, followed by wash and equilibration steps during which data was not acquired.

The LC was coupled to a timsTOF Pro MS (Bruker Daltonics) with a CaptiveSpray ion source. The MS was operated in parallel accumulation-serial fragmentation (PASEF) mode with dynamic exclusion, 10 PASEF MS/MS scans per 1.1 s cycle. Active exclusion for precursor ions was selected to release after 0.40 min, and precursors were reconsidered for analysis if the current intensity/previous intensity was greater than 4.0. The mass range was set between 100 and 1700 m/z, and the ion mobility range was set between 0.60 and 1.60 V·s/cm² with a ramp time of 100.0 ms. The intensity threshold was set to 5000 units and the target intensity was set to 20000 units. The collision energy was ramped between 20.00 to 59.00 eV as a function of ion mobility.

### Example 6: Data Processing and Statistical Analysis

The acquired raw data from each LC-MS analysis were imported onto PEAKS X Studio (Bioinformatics Solutions Inc., Waterloo, ON) for peptide identifications. The selected protein database for Human sapiens was downloaded from UniProt. The following parameters were employed for de novo peptide sequencing and protein identification via database search: precursor mass error tolerance=20 ppm, fragment mass error tolerance=0.05 Da, enzyme=trypsin, digest mode=specific, peptide length was set between 6 and 45 residues, and the maximum number of missed cleavages was set to 3. Carbamidomethylation of cysteine residues was set as a fixed modification hexose of lysine residues was set as a variable modification. Search result filters were set to a peptide discovery rate (FDR) of 1% and a protein filter was set to a −10 logP value>20. One of most frequently glycated albumin site, Lysine 525 (K_{(+162.0528)}QTALVELVK (SEQ ID NO:1), was used in peptide relative quantification. The GA level was calculated using the peak areas of K_{(+162.0528)}QTALVELVK (SEQ ID NO:1), signal divided by the sum of the peak areas of K_{(+162.0528)}QTALVELVK(SEQ ID NO:1), KQTALVELVK (SEQ ID NO:1), and QTALVELVK (SEQ ID NO:2), signals. The normal distribution of the data was assessed using the Shapiro-Wilk test, while the paired sample t-test or Wilcoxon signed-rank test was utilized to assess the intervention effects within the group.

### Example 7: Results

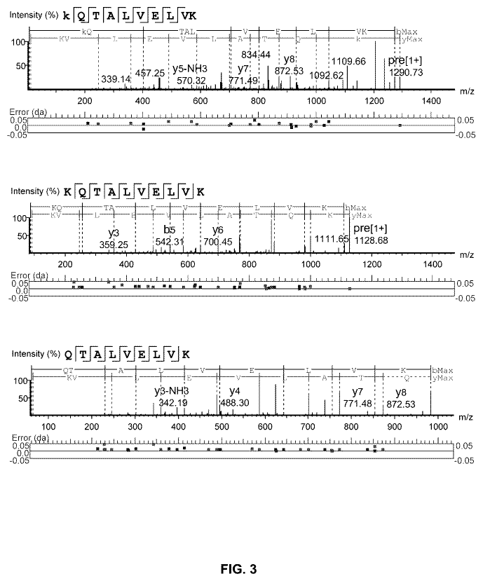

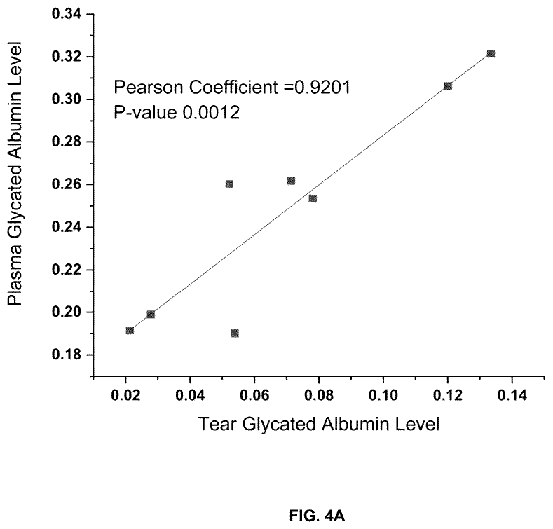

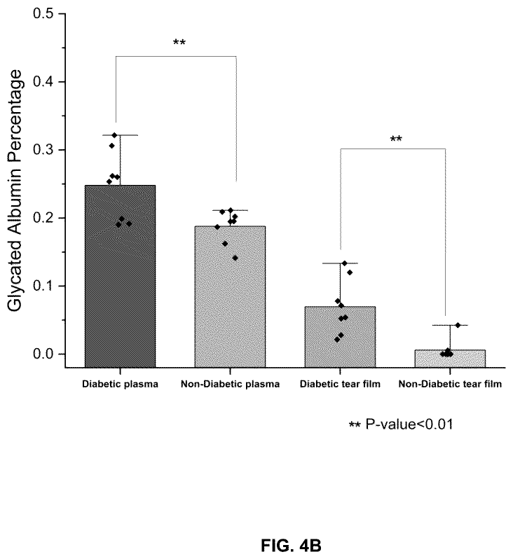

All plasma and tear samples were analyzed to confirm the presence of the peptides with high confidence m/z fragment measurements, as evidenced by the almost complete sequence coverage by the y fragment ion series (FIG. 3). The GA levels were significantly higher in diabetes-affected plasma and tears compared to controls with a p-value<0.01 (FIG. 4A). Lysine 525 glycation is not detected for most of the non-diabetic tear samples. A linear fit was performed for glycated albumin levels in the plasma and tear film diabetes samples (Table 1), and a strong correlation (Pearson's r=0.92019 with a P-value 0.0012) was observed for the plasma and tear film (FIG. 4B). Duplicates runs for all the plasma samples demonstrated good instrument reproducibility and robustness of quantitation method. The tear film samples could not be reproduced due to a small sample volume.

TABLE 1
Assessing the correlation of glycated albumin
level between plasma and tear film.

| Response | | Standard | | |
|---|---|---|---|---|
| variable | Value | Error | t-Value | Prob > \|t\| |
| Intercept | 0.16673 | 0.01602 | 10.40526 | 4.61591E−5 |
| Slope | 1.16382 | 0.20213 | 5.75763 | 0.0012 |

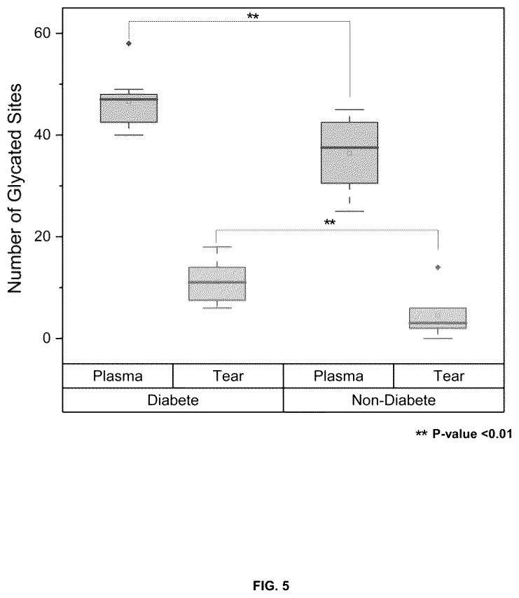

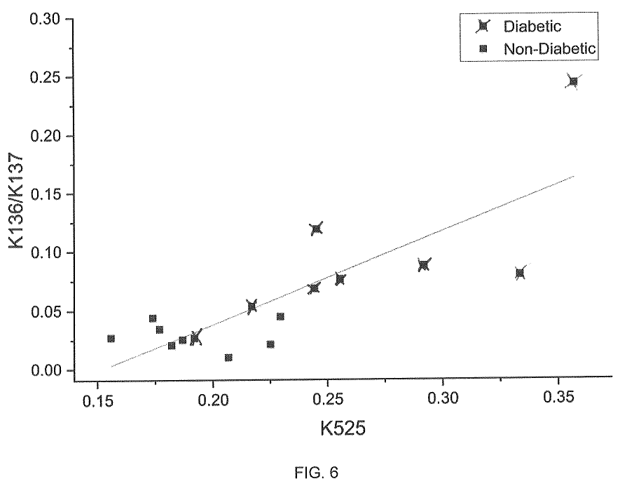

GA glycation occurs at Lys-525 and at other residues. The number of GA glycation sites was significantly higher in diabetes-affected plasma and tear comparatively to controls. Higher GA levels coexist with the larger number of glycation sites (FIG. 5). Moreover, levels of GA with Lysine 136/Lysine 137 (LVRPEVDVMCTAFHDNEETELK_{(+162.0528)} K_{(+162.0528)} (SEQ ID NO:25)) modification strongly correlate (Pearson coefficient 0.7987 with a P-value 0.00021) with the levels of GA modified at Lysine 525 residue (FIG. 6), suggesting presence of an alternative diabetes biomarker in plasma.

### Example 8: Discussion

In the present study, the principal glycation site of albumin in diabetic and non-diabetic participants is Lys-525 (K_{(+162.0528)}QTALVELVK (SEQ ID NO:1). The relative GA level in plasma and tear in both diabetes and non-diabetes was quantified. A significant higher GA level was found in diabetes in both plasma and tear film. We determined GA level between plasma and tear film is strongly correlated in diabetic patients. This is the first study to report such correlation. Based on this strong correlation, GA levels monitoring in tear is a promising approach for a quick and non-invasive characterization of diabetes-induced systemic effects.

Obtaining reproducible and unaltered tear samples can be challenging due to the limited volume of tears available for collection and the changing dynamic of the tear with reflex tears and blinking. There are several tear collection methods available for precorneal tear film analysis, but it is crucial to carefully evaluate the appropriate method to ensure the effectiveness of the assays and the quality of the results (e.g., non-stimulated tear sampling seems to be more appropriate and beneficial²⁰). Fullard and Snyder pointed out that the quantitative composition of the tear sample is significantly affected by the dilution caused by reflex tearing, emphasizing the need to regulate the tear flow rate when collecting tears.²¹ To address the issue, capillary tube tear collection method was used in this study, with the advantage over Schirmer's strips that is considered less invasive and avoids dilution caused by reflex tears as well as issues with extracting the fluid from the tissue.²² A 30 kDa MWCO filter was used to preconcentrate albumin before protein digestion as the abundant tear proteins/peptides below 30 kDa such as cystatin-S and prospholipase A2 as well as salts were removed. This process efficiently enhanced the sensitivity of downstream analysis, as it reduced sample complexity and minimized ion suppression effects. Given a total peptide amount 100 ng for the tear film sample, nanoLC offered extremely high sensitivity for such small volumes of diluted samples and reduced waste, in addition that TIMS offered an additional dimension of separation and collisional cross-section information, whereas PASEF greatly increases the preconcentration efficacy of albumin peptides. As a result, an average 95% albumin coverage was observed in tear film samples, with different glycation sites being detected.

HbA1c includes only one glycation site at the N-terminal valine of the β-chain of hemoglobin, but GA has multiple glycation sites. The present study showed that the number of GA glycation sites was significantly higher in diabetes-affected plasma and tear comparatively to controls. This indicates that GA level might reflect not only glycemic control but also structural and biochemical properties of albumin, and the structural changes influence the extent of glycation by way of catalysis of the Amadori rearrangement. Moderate glycations that occur naturally in vivo do not have a significant impact on its secondary structure due to the stable nature of albumin with high thermal unfolding temperatures, but glycation does affect the binding of ligands and drug molecules to albumin, which can be attributed to direct modification of lysine or arginine residues involved in drug binding or subtle structural changes that impact the architecture of a drug-binding site. A study utilizing albumin isolated from diabetic patients found a statistically significant reduction in the binding affinity for ketoprofen compared to albumin from normoglycemic individuals²⁵, highlighting the important role of albumin structural changes upon glycation in the variability of drug response in a diabetic situation.

The reactivity of individual lysine side chains towards glycation can vary depending on the local environment within the folded protein. Some lysine residues may have altered pKa values based on neighboring residues, and others may form strong hydrogen-bonds with nearby side chains or the peptide backbone. Lys-525 is considered to be the predominant site of the human serum albumin in vivo glycation, which is also the peptide being used for correlation analysis in this present study. Discovery of robust, selective, and specific biomarkers are important for early diagnosis and monitor diabetes-induced systemic effects. Levels of GA with Lys-136 or 137 modifications were found strongly correlated with the levels of GA modified at Lys-525 residue, suggesting presence of an alternative diabetes biomarker in plasma.

Glucose level can be predictive for both type 1 and type 2 diabetes in tear film. However, identifying a method to measure glucose levels in the eye without causing damage to the sclera that could lead to inaccurately high readings, while also obtaining consistent tear samples in sufficient volumes, has been the most significant challenge for measuring glucose in the tear film. The literature demonstrated spuriously high tear glucose determinations with use of paper strip collection methods that contact the conjunctiva, therefore, the method of tear collection and stimulation of tearing would significantly affect the result in the concentration of glucose.²⁸ To address this issue, Aihara et al.¹⁷ used a non-invasive tear collection method without stimulation of tears and demonstrated an association between tear and blood glucose concentrations. However, blood glucose level is known to be affected by food intake, and the 15 min time delay of glucose level between blood and tear can affect the stability and correlation of the glucose level obtains. ³⁰ In contrast, GA is not affected by food intake and reflects glycemic control status over to 2-3 weeks. Therefore, the correlation between GA level in tear and plasma would be more reliable for long-term glycemic monitor due to a relatively stable level of GA.

### Example 9: Conclusions

The results of the measurements showed that glycated albumin levels were significantly higher in diabetes-affected plasma and tears compared to controls with a p-value<0.01. A strong correlation of glycated albumin levels was observed for the plasma and tear film in diabetes samples (Pearson coefficient 0.92 with a p-value 0.0012). Therefore, GA detection in the tear fluid can be used as a non-invasive method for testing diabetic patients.

### REFERENCE

(1) Freitas, P. A. C.; Ehlert, L. R.; Camargo, J. L. Glycated Albumin: A Potential Biomarker in Diabetes. Arch. Endocrinol. Metab. 2017, 61 (3), 296-304. https://doi.org/10.1590/2359-3997000000272.
(2) Roglic, G. WHO Global Report on Diabetes: A Summary. Int. J. Noncommunicable Dis. 2016, 1 (1), 3. https://doi.org/10.4103/2468-8827.184853.
(3) Anguizola, J.; Matsuda, R.; Barnaby, O. S.; Hoy, K. S.; Wa, C.; DeBolt, E.; Koke, M.; Hage, D. S. Review: Glycation of Human Serum Albumin. Clin. Chim. Acta 2013, 425, 64-76. https://doi.org/10.1016/j.cca.2013.07.013.
(4) Zendjabil, M. Glycated Albumin. Clin. Chim. Acta 2020, 502, 240-244. https://doi.org/10.1016/j.cca.2019.11.007.
(5) Sherwani, S. I.; Khan, H. A.; Ekhzaimy, A.; Masood, A.; Sakharkar, M. K. Significance of HbA1c Test in Diagnosis and Prognosis of Diabetic Patients. Biomark. Insights 2016, 11, BMI.S38440. https://doi.org/10.4137/BMI.S38440.
(6) Zendjabil, M. Biological Diagnosis of Diabetes Mellitus. Curr. Res. Transl. Med. 2016, 64 (1), 49-52. https://doi.org/10.1016/j.patbio.2015.10.002.
(7) Kohzuma, T.; Tao, X.; Koga, M. Glycated Albumin as Biomarker: Evidence and Its Outcomes. J. Diabetes Complications 2021, 35 (11), 108040. https://doi.org/10.1016/j.jdiacomp.2021.108040.
(8) Shimizu, I.; Hiramatsu, Y.; Omori, Y.; Nakabayashi, M.; the JGA (Japan Glycated Albumin) Study Group. Comparison of HbA1c and Glycated Albumin as a Control Marker for Newborn Complications in Diabetic Women in a Multicentre Study in Japan (Japan Glycated Albumin Study Group: Study 2). Ann. Clin. Biochem. Int. J. Lab. Med. 2018, 55 (6), 639-646. https://doi.org/10.1177/0004563218763695.
(9) Nathan, D. M.; McGee, P.; Steffes, M. W.; Lachin, J. M.; the DCCT/EDIC Research Group. Relationship of Glycated Albumin to Blood Glucose and HbA1c Values and to Retinopathy, Nephropathy, and Cardiovascular Outcomes in the DCCT/EDIC Study. Diabetes 2014, 63 (1), 282-290. https://doi.org/10.2337/db13-0782.
(10) Hagan, S.; Martin, E.; Enriquez-de-Salamanca, A. Tear Fluid Biomarkers in Ocular and Systemic Disease: Potential Use for Predictive, Preventive and Personalised Medicine. EPMA J. 2016, 7 (1), 15. https://doi.org/10.1186/s13167-016-0065-3.
(11) You, J.; Willcox, M. D.; Madigan, M. C.; Wasinger, V.; Schiller, B.; Walsh, B. J.; Graham, P. H.; Kearsley, J. H.; Li, Y. Tear Fluid Protein Biomarkers. In Advances in Clinical Chemistry; Elsevier, 2013; Vol. 62, pp 151-196. https://doi.org/10.1016/B978-0-12-800096-0.00004-4.
(12) Farandos, N. M.; Yetisen, A. K.; Monteiro, M. J.; Lowe, C. R.; Yun, S. H. Contact Lens Sensors in Ocular Diagnostics. Adv. Healthc. Mater. 2015, 4 (6), 792-810. https://doi.org/10.1002/adhm.201400504.
(13) Dartt, D. A.; Willcox, M. D. P. Complexity of the Tear Film: Importance in Homeostasis and Dysfunction during Disease. Exp. Eye Res. 2013, 117, 1-3. https://doi.org/10.1016/j.exer.2013.10.008.
(14) Zhao, Z.; Liu, J.; Shi, B.; He, S.; Yao, X.; Willcox, M. D. P. Advanced Glycation End Product (AGE) Modified Proteins in Tears of Diabetic Patients. Mol. Vis. 2010.
(15) Terhaar, H. M.; Alibaugh, R. A.; Mochel, J. P.; Sebbag, L. Serum Albumin and Total Protein Concentration in the Tear Film of Horses with Healthy or Diseased Eyes. Vet. Ophthalmol. 2021, 24 (1), 20-27. https://doi.org/10.1111/vop.12822.
(16) Lane, J. D.; Krumholz, D. M.; Sack, R. A.; Morris, C. Tear Glucose Dynamics in Diabetes Mellitus. Curr. Eye Res. 2006, 31 (11), 895-901. https://doi.org/10.1080/02713680600976552.
(17) Aihara, M.; Kubota, N.; Minami, T.; Shirakawa, R.; Sakurai, Y.; Hayashi, T.; Iwamoto, M.; Takamoto, I.; Kubota, T.; Suzuki, R.; Usami, S.; Jinnouchi, H.; Aihara, M.; Yamauchi, T.; Sakata, T.; Kadowaki, T. Association between Tear and Blood Glucose Concentrations: Random Intercept Model Adjusted with Confounders in Tear Samples Negative for Occult Blood. J. Diabetes Investig. 2021, 12 (2), 266-276. https://doi.org/10.1111/jdi.13344.
(18) Aihara, M.; Kubota, N.; Kadowaki, T. Development of the Noninvasive Diabetes Monitoring Method. Presented at: European Association for the Study of Diabetes. Sep. 24, 2020; Stockholm, Sweden.
(19) Esmaeelpour, M.; Watts, P. O.; Boulton, M. E.; Cai, J.; Murphy, P. J. Tear Film Volume and Protein Analysis in Full-Term Newborn Infants. Cornea 2011, 30 (4), 400-404. https://doi.org/10.1097/ICO.Ob0l3e3181f22cd9.
(20) Rentka, A.; Koroskenyi, K.; Harsfalvi, J.; Szekanecz, Z.; Szucs, G.; Szodoray, P.; Kemeny-Beke, A. Evaluation of Commonly Used Tear Sampling Methods and Their Relevance in Subsequent Biochemical Analysis. Ann. Clin. Biochem. Int. J. Lab. Med. 2017, 54 (5), 521-529. https://doi.org/10.1177/0004563217695843.
(21) Fullard, R. J.; Snyderj, C. Protein Levels in Nonstimulated and Stimulated Tears of Normal Human Subjects. No. 6.
(22) Nandi, S. K.; Singh, D.; Upadhay, J.; Gupta, N.; Dhiman, N.; Mittal, S. K.; Mahindroo, N. Identification of Tear-Based Protein and Non-Protein Biomarkers: Its Application in Diagnosis of Human Diseases Using Biosensors. Int. J. Biol. Macromol. 2021, 193, 838-846. https://doi.org/10.1016/j.ijbiomac.2021.10.198.
(23) Kisugi, R.; Kouzuma, T.; Yamamoto, T.; Akizuki, S.; Miyamoto, H.; Someya, Y.; Yokoyama, J.; Abe, I.; Hirai, N.; Ohnishi, A. Structural and Glycation Site Changes of Albumin in Diabetic Patient with Very High Glycated Albumin. Clin. Chim. Acta 2007, 382 (1-2), 59-64. https://doi.org/10.1016/j.cca.2007.04.001.
(24) Vetter, S. W. Glycated Serum Albumin and AGE Receptors. In Advances in Clinical Chemistry; Elsevier, 2015; Vol. 72, pp 205-275. https://doi.org/10.1016/bs.acc.2015.07.005.
(25) Baraka-Vidot, J.; Planesse, C.; Meilhac, O.; Militello, V.; Bourdon, E.; Rondeau, P. Glycation Alters Ligand Binding, Enzymatic, and Pharmacological Properties of Human Albumin. 2015.
(26) Gadgil, H. S.; Bondarenko, P. V.; Treuheit, M. J.; Ren, D. Screening and Sequencing of Glycated Proteins by Neutral Loss Scan LC/MS/MS Method. Anal. Chem. 2007, 79 (15), 5991-5999. https://doi.org/10.1021/ac070619k.
(27) Priego-Capote, F.; Scherl, A.; Möller, M.; Waridel, P.; Lisacek, F.; Sanchez, J.-C. Glycation Isotopic Labeling with 13C-Reducing Sugars for Quantitative Analysis of Glycated Proteins in Human Plasma. Mol. Cell. Proteomics 2010, 9 (3), 579-592. https://doi.org/10.1074/mcp.M900439-MCP200.
(28) Baca, J. T.; Finegold, D. N.; Asher, S. A. Tear Glucose Analysis for the Noninvasive Detection and Monitoring of Diabetes Mellitus. Ocul. Surf. 2007, 5 (4), 280-293. https://doi.org/10.1016/Si542-0124(12)70094-0.
(29) Sen, D. K.; Sarin, G. S. Tear Glucose Levels in Normal People and in Diabetic Patients. Br. J. Ophthalmol. 1980, 64 (9), 693-695. https://doi.org/10.1 136/bjo.64.9.693.
(30) Sempionatto, J. R.; Brazaca, L. C.; Garcia-Carmona, L.; Bolat, G.; Campbell, A. S.; Martin, A.; Tang, G.; Shah, R.; Mishra, R. K.; Kim, J.; Zucolotto, V.; Escarpa, A.; Wang, J. Eyeglasses-Based Tear Biosensing System: Non-Invasive Detection of Alcohol, Vitamins and Glucose. Biosens. Bioelectron. 2019, 137, 161-170. https://doi.org/10.1016/j.bios.2019.04.058.
(31) Sitaramamma, T.; Shivaji, S.; Rao, G. N. Effect of Storage on Protein Concentration of Tear Samples. Curr. Eye Res. 1998, 17 (10), 1027-1035. https://doi.org/10.1076/ceyr.17.10.1027.5241.
(32) Hamm-Alvarez, S. F.; Okamoto, C. T.; Janga, S. R.; Feigenbaum, D.; Edman, M. C.; Freire, D.; Shah, M.; Ghanshani, R.; Mack, W. J.; Lew, M. F. Oligomeric α-Synuclein Is Increased in Basal Tears of Parkinson's Patients. Biomark. Med. 2019, 13 (11), 941-952. https://doi.org/10.2217/bmm-2019-0167.
(33) Wang, F.; Ma, X.; Hao, Y.; Yang, R.; Ni, J.; Xiao, Y.; Tang, J.; Bao, Y.; Jia, W. Serum Glycated Albumin Is Inversely Influenced by Fat Mass and Visceral Adipose Tissue in Chinese with Normal Glucose Tolerance. PLoS ONE 2012, 7 (11), e51098. https://doi.org/10.1371/journal.pone.0051098.

1. A biosensor for the detection of glycated albumin in a biological sample, the biosensor comprising: a carbon printed layer, a layer of a ligands that can specifically bind glycated albumin in contact with the carbon printed layer; at least one sensor electrode; and a substrate.

2. The biosensor of claim 1, wherein the at least one sensor electrode is between the carbon-printed layer and the layer of a ligand for the analyte.

3. The biosensor of claim 1, wherein the ligands are antibodies or aptamers that can specifically bind glycated albumin.

4. The biosensor of claim 1, wherein osmolarity is detected in combination with glycated albumin.

5. (canceled)

6. A method for detecting glycated albumin in a sample and osmolarity of the sample, comprising contacting the biosensor of claim 1 with the sample and detecting the glycated albumin and the osmolarity with the detector.

7. The method of claim 6, wherein the detector detects binding or interaction of the glycated albumin and the ligands due to a change in electrical impedance caused by binding or interaction of the glycated albumin in the sample with the ligands.

8. The method of claim 6, wherein the sample is tears, tear film, aqueous layer of the tear film, aqueous humor, sweat, blood, serum, plasma, urine, or saliva.

9. The method of claim 6, wherein the sample size is between about 1 μL and 5 μL.

10. (canceled)

11. A method for detecting glycated albumin in a sample and osmolarity of the sample, comprising contacting the sample with the biosensor of claim 1, and inserting the biosensor into or onto a device comprising (a) a detector to measure change in electrical impedance, and (b) a screen for visualizing the change in impedance, thereby detecting one or more analytes in the sample and the osmolarity of the sample.

12. The method of claim 11, wherein an amount of the glycated albumin in the sample is detected.

13. (canceled)

14. A method for detecting glycated albumin in a sample and osmolarity of the sample, comprising contacting the sample with the biosensor of claim 1, wherein the biosensor is present in a handheld device comprising (a) a detector to measure change in electrical impedance, and (b) a screen for visualizing the change in impedance, thereby detecting one or more analytes in the sample and the osmolarity of the sample.

15. The method of claim 14, wherein an amount of the glycated albumin in the sample is detected.

16. (canceled)

17. A method for diagnosing diabetes, comprising contacting the biosensor of claim 1 with a tear, tear film, aqueous layer of the tear film, or aqueous humor sample of a subject, detecting an amount glycated albumin in the sample and osmolarity of the sample, and diagnosing diabetes in the subject where the concentration of the glycated albumin in the sample is elevated as compared to a control sample or control standard and/or wherein the osmolarity of the sample is elevated as compared to a control sample or control element.

18. The method of claim 17, wherein an increased amount of osmolarity as compared to a control indicates a diagnosis of diabetes.

19. The method of claim 17, further comprising treating the subject with bolus insulin, rapid-acting insulin, inhaled insulin, short-acting insulin, background insulin, intermediate-acting insulin, long-acting insulin, insulin glargine, insulin degludec, premixed insulin, biguanides, DPP-4 inhibitors, GLP-1 receptor agonists, sodium-glucose cotransporter-2 (SGLT2) inhibitors, insulin secretagogues, meglitinides, thiazolidinediones, vascular endothelial growth factor inhibitors, photocoagulation laser treatment, panretinal photocoagulation laser treatment, vitrectomy, or a combination thereof.

20. (canceled)

21. The method of claim 1, wherein the sample is obtained from a subject having end-stage renal disease, liver cirrhosis, iron deficiency, or anemia.

22. The method of claim 21, wherein the anemia is hemolytic anemia or renal anemia.

23. The method of claim 1, wherein the sample is obtained from a subject is not required to be fasted.

24. A handheld device comprising a biosensor comprising a carbon printed layer, a layer of a ligands specific for glycated albumin in contact with the carbon printed layer; at least one sensor electrode; and a substrate; and a detector connected to a data acquisition system.

25. The handheld device of claim 24, wherein the detector is a multimeter.

26. (canceled)

27. (canceled)


SEQUENCE LISTING (25 sequences)

SEQ ID NO: 1 - protein, 10 aa, synthetic construct
KQTALVELVK

SEQ ID NO: 2 - protein, 9 aa, synthetic construct
QTALVELVK

SEQ ID NO: 3 - DNA, 59 bp, synthetic construct
  source 1..59: mol_type = other DNA
atgcggatcccgcgcgcagtgcagggagccgctccacgtacgttgcgcgaagcttgcgc

SEQ ID NO: 4 - DNA, 23 bp, synthetic construct
  source 1..23: mol_type = other DNA
  modified_base 1: mod_base = OTHER; note = 6-(4-Monomethoxytritylamino)hexyl-(2-cyanoethyl)-(N ,N-diisopropyl)-phosphoramidite modified nucleotide
  modified_base 23: mod_base = OTHER; note = 1-O-Dimethoxytrityl-hexyl-disulfide,1'-[(2-cyanoeth yl)-(N,N-diisopropyl)]-phosphoramidite modified nucleotide
tgcggttgtagtactcgtggccg

SEQ ID NO: 5 - DNA, 33 bp, synthetic construct
  source 1..33: mol_type = other DNA
tctatccccccagccttcccactccaaccctgc

SEQ ID NO: 6 - DNA, 38 bp, synthetic construct
  source 1..38: mol_type = other DNA
cgggccgctgcatataactgaattgaataagctggtat

SEQ ID NO: 7 - DNA, 52 bp, synthetic construct
  source 1..52: mol_type = other DNA
tggtacatcgaccatcaccgcacctcacatattccgaattactcccgacgta

SEQ ID NO: 8 - DNA, 52 bp, synthetic construct
  source 1..52: mol_type = other DNA
tacattgctcctgcggaaaaattgtcaaaccatctactgcgaagcgtgtttt

SEQ ID NO: 9 - DNA, 49 bp, synthetic construct
  source 1..49: mol_type = other DNA
taggagtagggggtcgtagacggttggggcggaacgggcgtggggcatg

SEQ ID NO: 10 - DNA, 53 bp, synthetic construct
  source 1..53: mol_type = other DNA
tggtacatcgaccatcaccgcacctcacatattccgaattactcccgacgtat

SEQ ID NO: 11 - DNA, 53 bp, synthetic construct
  source 1..53: mol_type = other DNA
tcgatggtgggcagccccagcacattccgtatgttaacccctgcgttgccatt

SEQ ID NO: 12 - DNA, 49 bp, synthetic construct
  source 1..49: mol_type = other DNA
ggtgcggttcgtgcggttgtagtactcgtggccgatagaggtagtttcg

SEQ ID NO: 13 - DNA, 51 bp, synthetic construct
  source 1..51: mol_type = other DNA
tcatactgggtcatgtacttagctggtcgcagcggggactgagttagtgtt

SEQ ID NO: 14 - DNA, 53 bp, synthetic construct
  source 1..53: mol_type = other DNA
tcccacgcccgcccgtcgttcacccctccccgctacctccctatccaactgcg

SEQ ID NO: 15 - DNA, 53 bp, synthetic construct
  source 1..53: mol_type = other DNA
tccccccatcacacccaagccgcagccaccgacatagcaagcattgtctttcc

SEQ ID NO: 16 - DNA, 52 bp, synthetic construct
  source 1..52: mol_type = other DNA
tcgggggggcgttgattttgttgaagggaggtatagtgtctgtcggtctgat

SEQ ID NO: 17 - DNA, 51 bp, synthetic construct
  source 1..51: mol_type = other DNA
tcctgccgaactccaagatctccgctccgctcacgctgtgtatccatgggg

SEQ ID NO: 18 - DNA, 53 bp, synthetic construct
  source 1..53: mol_type = other DNA
tagttctaggccgccctcgtgataacccccctccatcttccctacgatgtact

SEQ ID NO: 19 - DNA, 52 bp, synthetic construct
  source 1..52: mol_type = other DNA
tgggtcatcgtcgtcttaggcgcgtgaaaggggtaggatggcgggtaggatg

SEQ ID NO: 20 - DNA, 52 bp, synthetic construct
  source 1..52: mol_type = other DNA
tgcaaggtgggcattggcattgcgtagctagggggtgaaggcgtgtggtttt

SEQ ID NO: 21 - DNA, 71 bp, synthetic construct
  source 1..71: mol_type = other DNA
tcaggcaaacacaatatacgcaatatcacggtggaatttcaaggcctttcatcaattgaataagctggtat

SEQ ID NO: 22 - DNA, 53 bp, synthetic construct
  source 1..53: mol_type = other DNA
tcaaaagcgcgctaagcctagttcgacaacttcaccaacgacccactattcgt

SEQ ID NO: 23 - DNA, 51 bp, synthetic construct
  source 1..51: mol_type = other DNA
tccctaacccgctctaaccaaccgcgctcagtccgacatccgtaaacgggc

SEQ ID NO: 24 - DNA, 53 bp, synthetic construct
  source 1..53: mol_type = other DNA
tccaacccagaccaacattcctcgcctccgctatctgcaccgccacacataac

SEQ ID NO: 25 - protein, 23 aa, synthetic construct
LVRPEVDVMCTAFHDNEETELKK